(19) United States
(12) Patent Application Publication
(10) Pub. No.: US 2007/0293788 A1
(43) Pub. Date: Dec. 20, 2007

(54) Bone harvest system

(75) Inventors: Dean Allen Entrekin, Downingtown, PA (US); Michael W. Paris, Lansdale, PA (US); Charanpreet S. Bagga, Phoenixville, PA (US); Donald L. Scanlan, Huntingdon Valley, PA (US); Todd F. Alamin, Woodside, CA (US); Lawrence T. Khoo, Studio City, CA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Vita Special Purpose Corporation, Wilmington, DE (US)

(21) Appl. No.: 11/471,394

(22) Filed: Jun. 19, 2006

(51) Int. Cl. A61B 10/00

(52) U.S. Cl. 600/564

(57) ABSTRACT

Harvesting apparatuses comprising a generally cylindrical outer cannula having one end secured in a first handle and having a distal end that is generally semicylindrical, and a generally cylindrical inner cannula having one end secured in a second handle and having a distal end that is generally semicylindrical, are provided. The present invention provides for apparatuses wherein the inner cannula is rotatable with respect to the outer cannula such that, in cooperation with the outer cannula, a volume at the distal end of both cannulas is substantially enclosed. Also provided are kits comprising the apparatuses of the present invention, as well as methods of using the apparatuses of the present invention.

### FIELD OF THE INVENTION

The present invention provides for apparatuses for the harvesting of bone and bone marrow aspirate. Also provided are kits and methods for using the apparatuses in retrieving bone and bone marrow aspirate.

### BACKGROUND OF THE INVENTION

Orthopedic, neurosurgical, spinal, ear-nose-throat, oralmaxillofacial, and rheumatology procedures typically require the harvesting of bone and/or bone cells for culturing or for placing in areas of the body to allow for fusion or new bone formation. Minimally-invasive devices that can harvest bone from a donor site with limited morbidity are thus desired.

### SUMMARY OF THE INVENTION

Bone harvesting apparatuses comprising a generally cylindrical outer cannula having one end secured in a first handle and having a distal end that is generally semicylindrical, and a generally cylindrical inner cannula having one end secured in a second handle and having a distal end that is generally semicylindrical, are provided. The present invention provides for apparatuses wherein the inner cannula is rotatable with respect to the outer cannula such that, in cooperation with the outer cannula, a volume at the distal end of both cannulas is substantially enclosed. Also provided are kits comprising the apparatuses of the present invention, as well as methods of using the apparatuses of the present invention.

### BRIEF DESCRIPTION OF THE DRAWINGS

FIG. 1A is an illustration of one embodiment of the system of the present invention.

FIG. 1B is an illustration of one embodiment of the system of the present invention.

FIG. 1C is an illustration of one embodiment of the system of the present invention.

FIG. 2A illustrates an exemplary inner cannula of the present invention.

FIG. 2B illustrates an exemplary outer cannula of the present invention.

FIG. 3A illustrates an exemplary inner cannula of the present invention.

FIG. 3B illustrates an exemplary outer cannula of the present invention.

FIGS. 4A-4B illustrate detailed views of exemplary distal ends of the inner and outer cannulas of the present invention in the open (FIG. 4A) and closed (FIG. 4B) positions.

FIGS. 5A-5C illustrate detailed views of exemplary distal ends of the inner and outer cannulas of the present invention.

FIGS. 6A-6C illustrate exemplary handles of the present invention, as viewed from the top.

FIG. 6D illustrates exemplary cooperating detents of the present invention

FIG. 6E illustrates a cross-sectional view of exemplary handles of the present invention, detailing one embodiment of cooperating detents of the present invention.

FIG. 7 illustrates one embodiment of the present invention wherein a harvested bone sample is ejected from the inner cannula.

FIG. 8 is an illustration of one embodiment of the plunger of the present invention.

FIG. 9 illustrates an exemplary fenestrated cannula, inserted into an outer cannula of the present invention.

FIG. 10 illustrates one embodiment of a fenestrated cannula of the present invention.

FIGS. 11A-11B illustrate detailed views of exemplary distal ends of the outer cannula and fenestrated cannula of the present invention.

FIG. 12A is an illustration of a transverse cross-section of one embodiment of the distal ends of the inner and outer cannulas of the present invention.

FIG. 12B is an illustration of a longitudinal cross-section of one embodiment of the distal ends of the inner and outer cannulas of the present invention.

### DETAILED DESCRIPTION OF ILLUSTRATIVE EMBODIMENTS

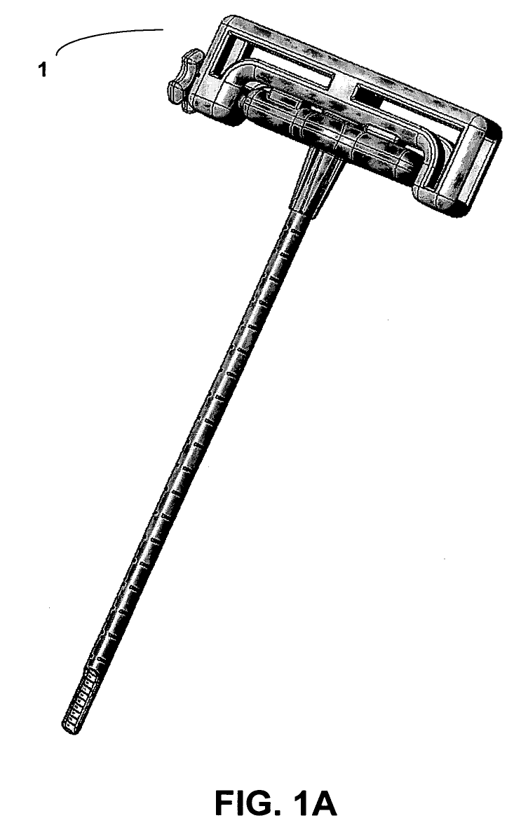

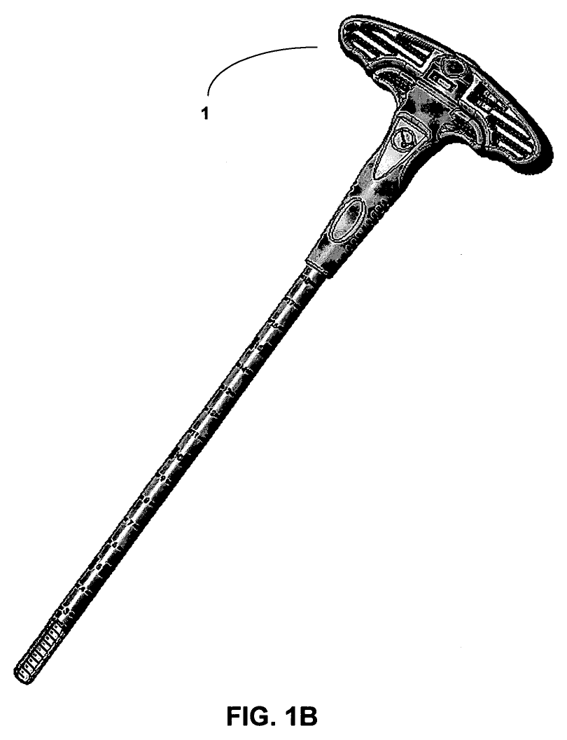

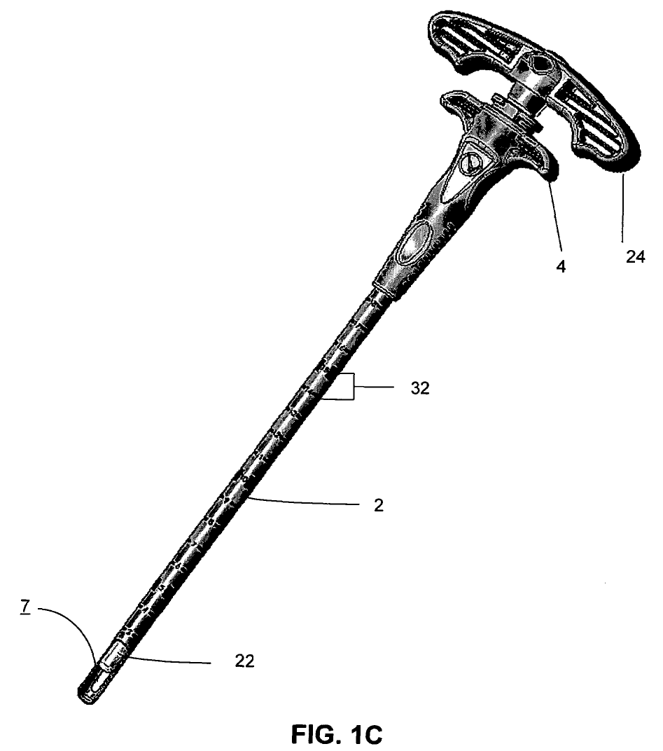

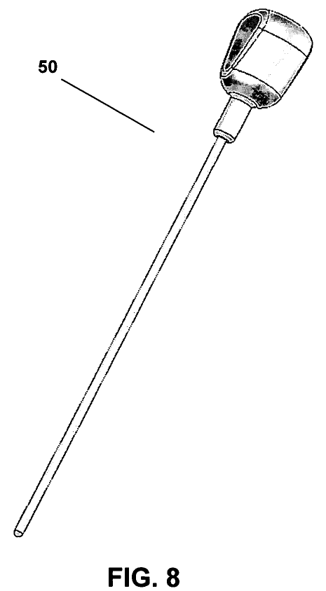

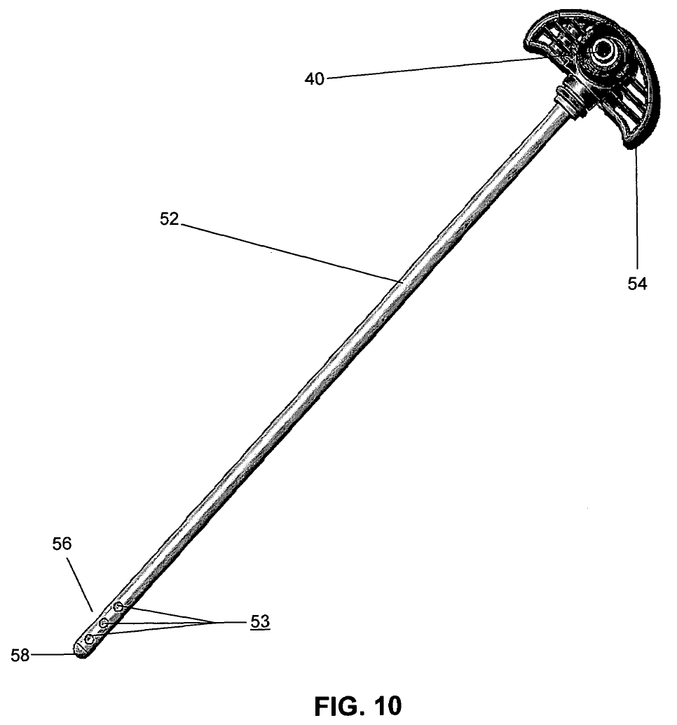

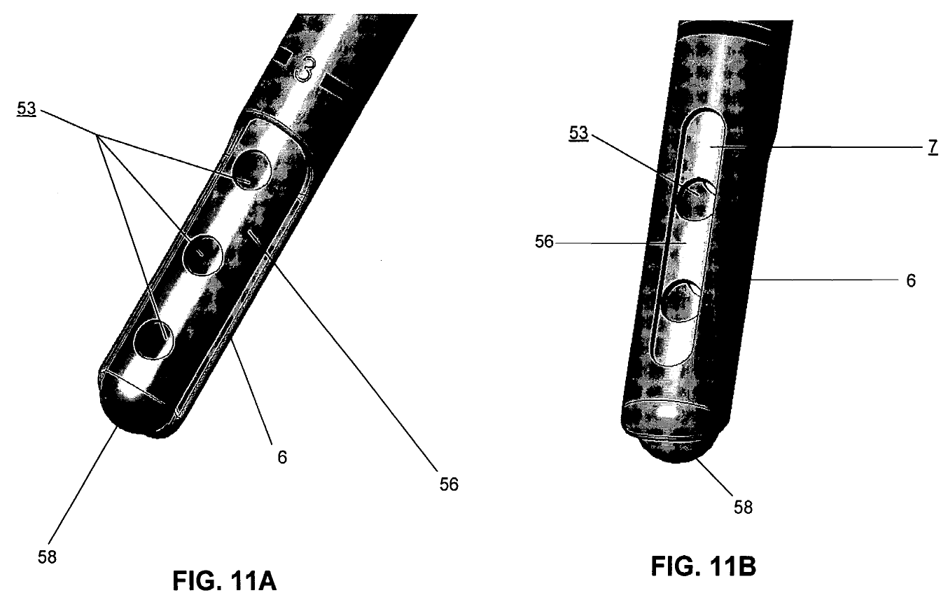

Referring to FIGS. 1-12, a harvesting system 1 has an outer cannula 2 having a distal end 6 that is generally semicylindrical. As used herein, semicylindrical is defined as having the shape of a longitudinal half of a cylinder. In an exemplary embodiment of the present invention, the tip 8 of the distal end 6 is generally rounded or generally convex. The tip 8 may also be generally blunt.

The distal end 6 may also comprise at least one aperture 7. It is preferred that the aperture 7 be elongated to align with fenestrations within a bone marrow aspirate cannula such as that described herein. It is also preferred that the aperture 7 be shaped so as to maintain the strength of the distal end of the cannula.

In other preferred embodiments, the outer cannula 2 may comprise graduated markings 32. These markings may be laser etched. It may be convenient for the gradations to be spaced 1 cm apart to allow for depth measurement within cancellous bone and body cavity spaces.

The proximal end of the outer cannula 2 may be secured in a first handle 4. The first handle 4 may be composed of a rigid medical grade plastic or any other rigid medical grade material, such as, for example, stainless steel. Preferably, the first handle 4 has a port 10, a port being defined as an opening through which other materials or objects may pass. Port 10 preferably serves for receiving the inner cannula or other such devices as described below.

A bone harvest system 1 also comprises an inner cannula 22 having a distal end 26 that is generally semicylindrical. As used herein, semicylindrical is defined as having the shape of a longitudinal half of a cylinder. In an exemplary embodiment of the present invention, the distal ends of the inner and outer cannulas have a semicircular circumference defined by an arc greater than about 180° for retaining harvested bone 60. In certain embodiments, the distal ends of the inner and outer cannulas are defined by an arc greater than about π radians. In an exemplary embodiment of the present invention, the circumference of the semicylindrical distal ends of the inner and outer cannulas is greater than about 50% of their total circumference. In another embodiment, the circumference of the distal end of the inner cannula is greater than the circumference of the distal end of the outer cannula. Preferably, the tip 28 of the distal end 26 is generally rounded or generally convex. The tip 28 of the distal end 26 may also be generally blunt. These generally rounded, generally convex, and generally blunt distal ends minimize the risk for accidental perforation of the cortical bone boundaries. Perforation of the cortical bone boundaries could result in damage to nerves, vessels, and/or soft tissue structures.

The distal end 26 may further comprise at least one cutting surface. It is preferred that each exposed edge 36 of the distal end 26 comprises a cutting surface.

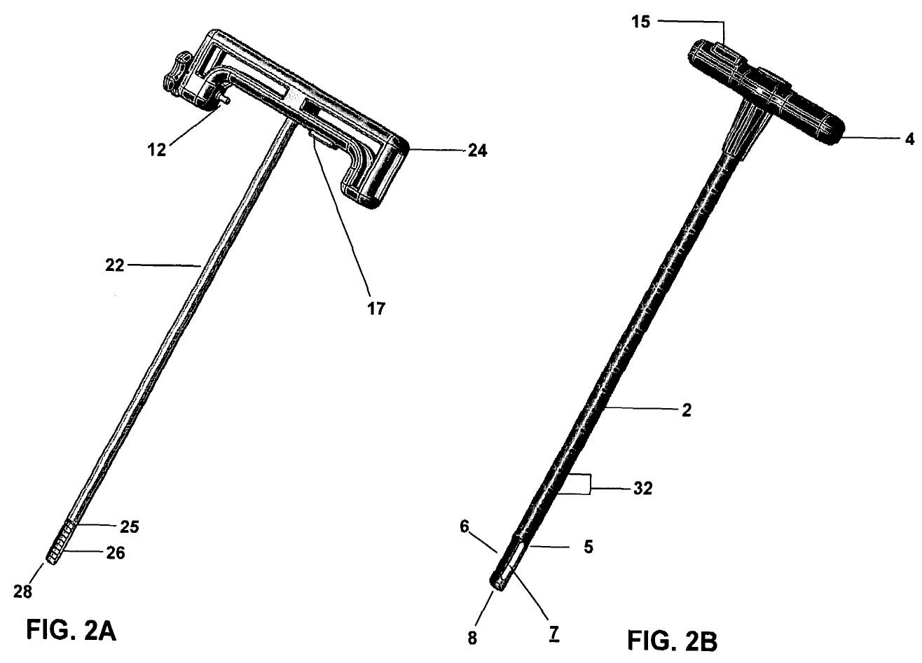

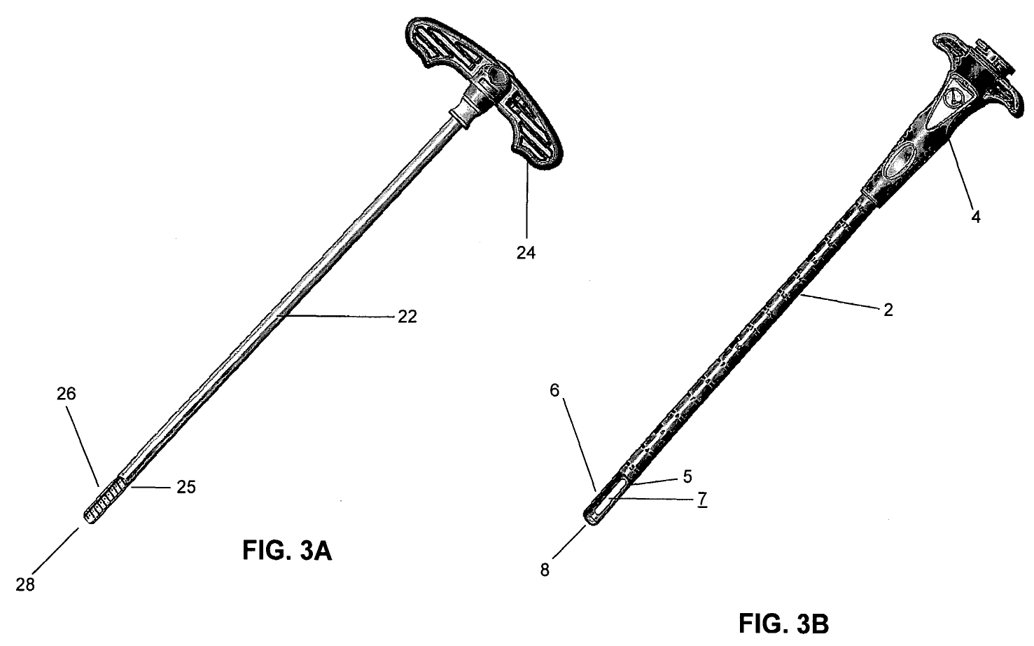

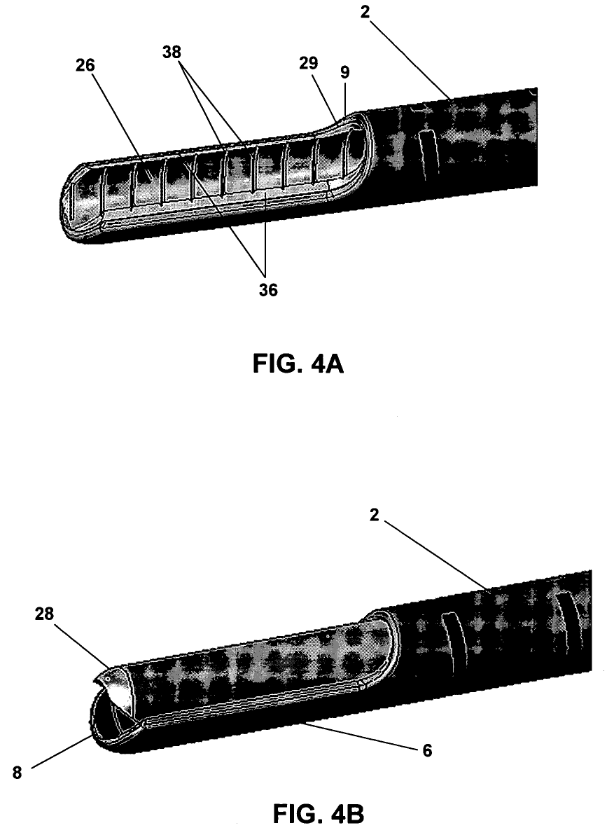

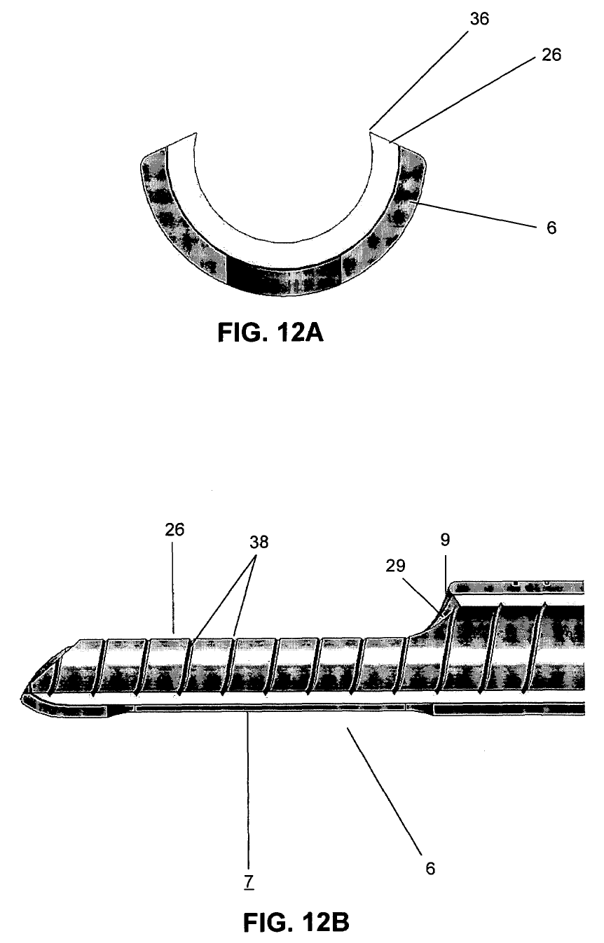

In certain embodiments, distal ends 6 and 26 comprise proximal surfaces 5 and 25, shown in, for example, FIGS. 2-3. As depicted in FIGS. 4B and 12B, the proximal surfaces of the inner and outer cannulas may further comprise angled chamfers (9, 29). Chamfers 9 and 29 align when the cannulas are in an open assembly to facilitate entry of harvested bone 60 into the interior of the inner cannula.

In a preferred apparatus of the present invention, the inner surface of distal end 26 may comprise a plurality of furrows 38. As used herein, furrows are defined as ruts, grooves, indentations, depressions, or trenches that can retain harvested bone material. In some embodiments, the furrows 38 are aligned normal to the longitudinal axis of the inner cannula 22. In others, the furrows are canted with respect to the transverse plane of the inner cannula 22.

Alternatively, the inner surfaces of both the inner cannula 22 and/or the outer cannula 2 may comprise a plurality of furrows 38. In some embodiments, the furrows 38 are aligned normal to the longitudinal axes of the cannulas. In others, the furrows 38 form a generally threaded pattern in the inner surfaces of the cannulas.

Cannulas 2 and 22 may be composed of any biocompatible, sterilizable material. It is preferred that the cannulas are composed of any medical grade material that has the strength and rigidity to perforate through cortical bone and navigate within the marrow space. The cannulas are preferably composed of stainless steel or rigid plastic material. The cannulas may also be composed of other metals such as medical-grade titanium.

In some embodiments of the present invention, the proximal end of the inner cannula 22 is secured in a second handle 24. The second handle 24 may be composed of a rigid medical grade plastic or any other rigid medical grade material, such as, for example, stainless steel. The second handle 24 may also have a port such as 10, a port being defined as an opening through which other materials or objects may pass.

One way of assembling a bone harvest system of the present invention is to insert inner cannula 22 into a port 10 of the first handle 4 and coaxially insert the inner cannula into the outer cannula 2. Preferably, the diameter of the inner cannula 22 is less than the diameter of the outer cannula 2. More preferably, the diameter of the inner cannula 22 is such that the inner cannula may be slidable and rotatable within the outer cannula 2. Most preferably, the diameter of the inner cannula 22 is the largest diameter possible that will allow for the inner cannula to be slidable and rotatable within the outer cannula 2.

The lengths of the inner and outer cannulas may be such that the tip 8 of the outer cannula 2 and the tip 28 of the inner cannula 22 are generally aligned when the inner cannula 22 is fully inserted into the outer cannula 2. Furthermore, the angled chamfers 9 and 29 align along their circumferences when the assembly is in an open configuration, so as to direct the volume of cancellous bone into the interior of the inner cannula. FIGS. 12A and 12B illustrate cross-sectional views of one embodiment of the distal ends of the inner and outer cannulas.

Preferably, the first handle 4 will be seated under the second handle 24 when the bone harvest system 1 is fully assembled. In an exemplary embodiment of the present invention, the first handle 4 and second handle 24 cooperate such that the second handle may be rotated with respect to the first handle so as to cause the inner cannula 22 to rotate within the outer cannula 2. More preferably, the first handle 4 and second handle 24 cooperate such that the second handle may be rotated with respect to the first handle so as to cause the inner cannula 22 to rotate within the outer cannula 2, resulting in the distal ends of the cannulas to substantially enclose a volume.

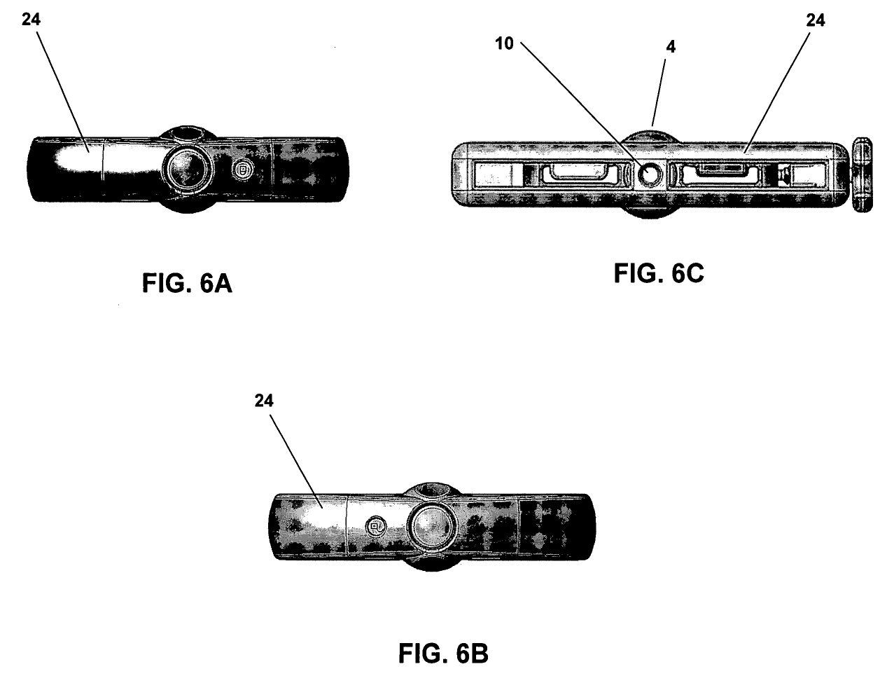

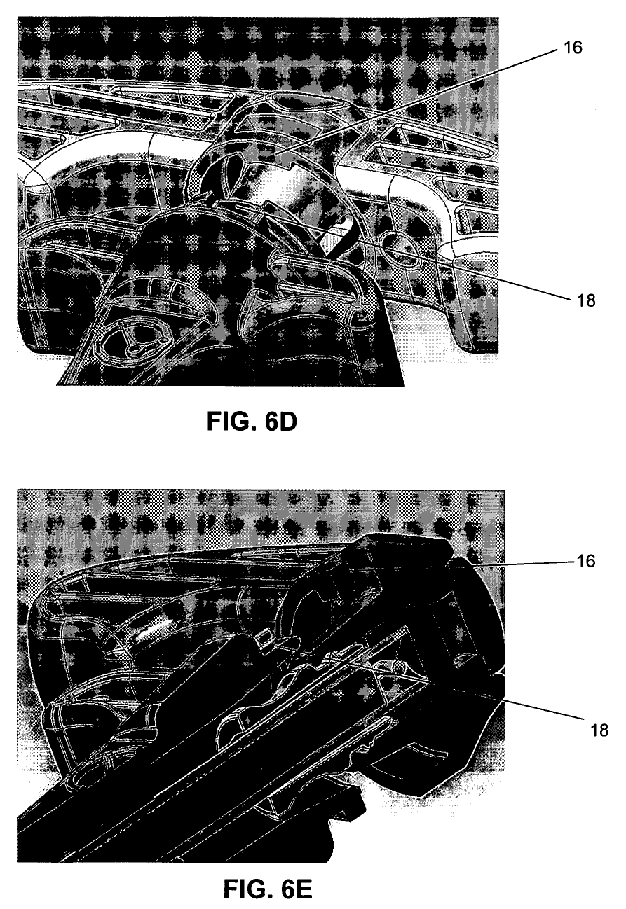

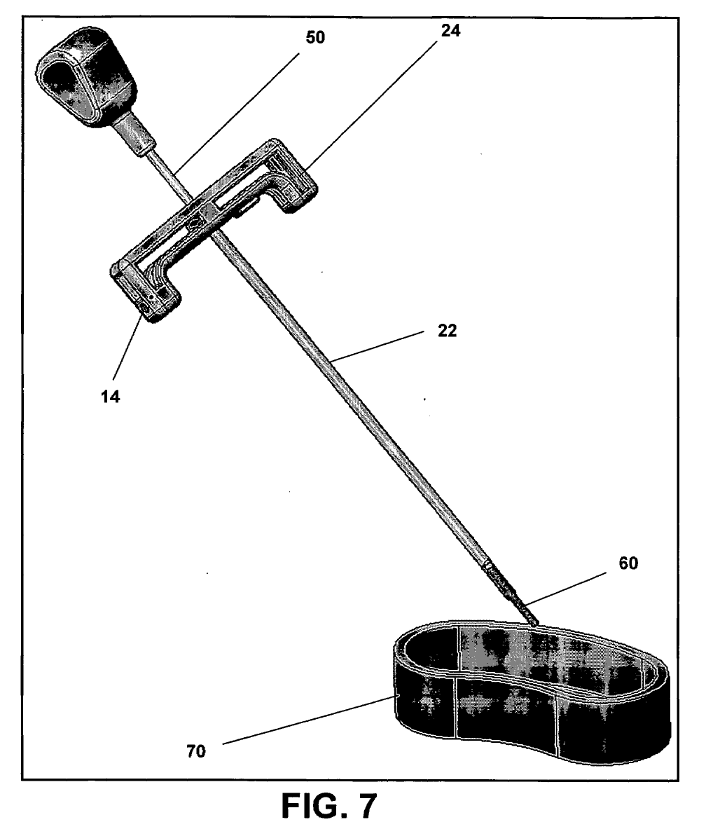

In some embodiments, the first and second handle may comprise mechanical means for locking the handles in a fixed position so as to prevent undesired movement of the inner cannula within the outer cannula when in an open assemble for insertion into cancellous bone. One mechanical means for locking, shown in FIG. 6D, may comprise cooperating detents (16, 18), a detent being defined as a device, such as a tab or catch, for positioning and holding one handle in relation to another so that the device can be released by force applied to one of the handles. In certain embodiments, the cooperating detents may comprise a tongue and groove-type assembly. Another mechanical means for locking may comprise the second handle 24 having a pin 12 and the first handle having an aperture 14 for receiving pin 12. In some embodiments the pin 12 may be a rod. In others, the pin 12 may be a screw. The aperture 14 may also be threaded.

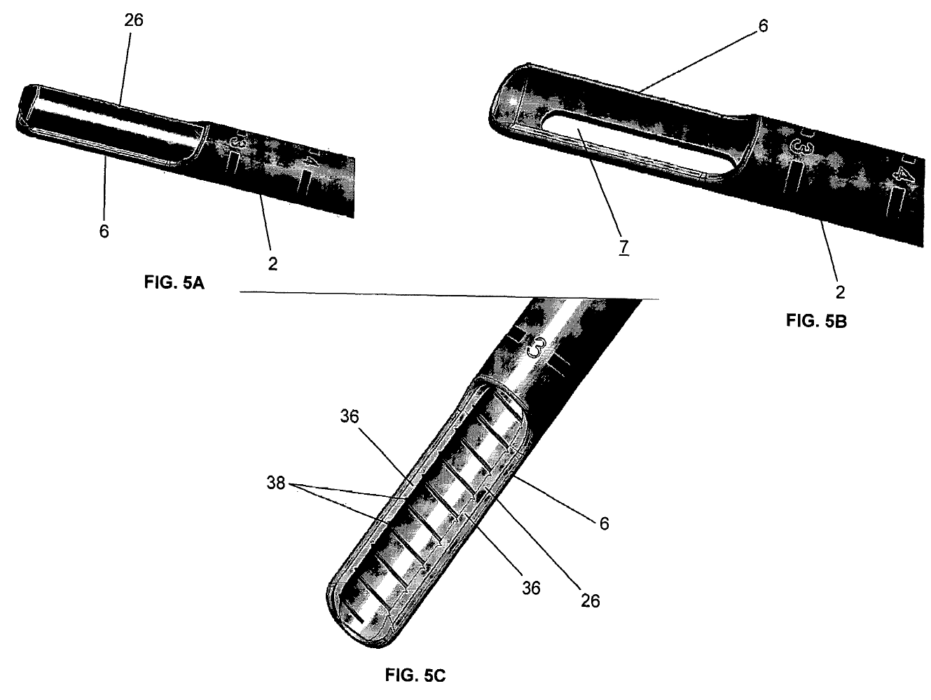

It is preferred that when the handles are in a locked position, the distal ends of the cannulas are in an “open” position, wherein the distal ends generally overlap (see FIGS. 4A and 5C).

In some embodiments, the first and second handle may further comprise mechanical means for restricting rotation of the inner cannula when assembled in the outer cannula. These mechanical means may comprise any device, such as a tab or catch, such that a rotation of greater than approximately 180° from the locked position is prevented. For example, such mechanical means may comprise tabs 17 and 15, shown in FIGS. 2A and 2B. In preferred embodiments, rotation of approximately 180° from the locked position corresponds to an assembly where the distal ends of the inner and outer cannulas are in a substantially closed configuration.

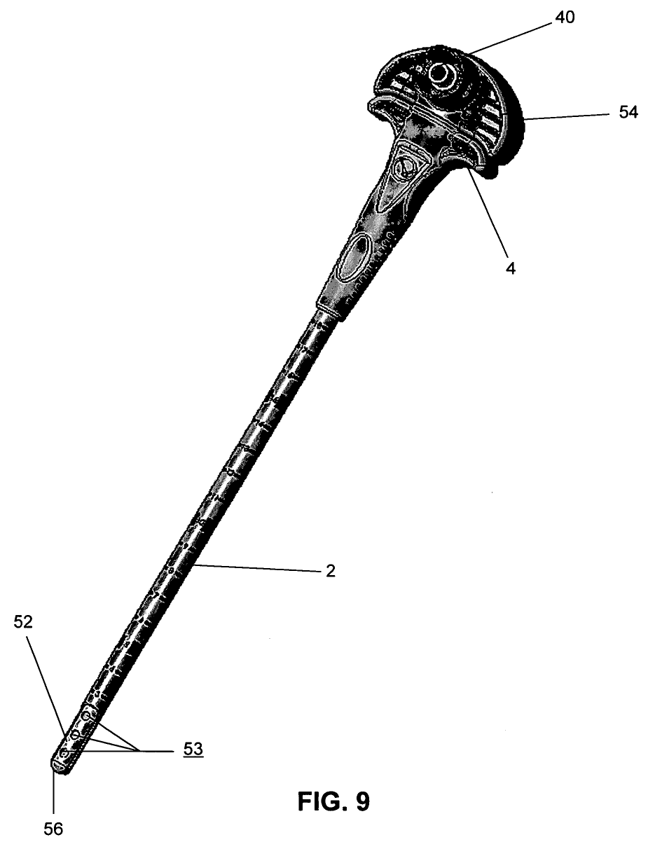

Referring to FIGS. 9-11, the system of the present invention may further comprise a fenestrated cannula 52 having a generally cylindrical distal end 56 comprising a plurality of fenestrations 53. The fenestrations may be generally aligned along the longitudinal plane of cannula 52. Preferably, the fenestrations are aligned such that at least some of the fenestrations are exposed through aperture 7 when cannula 52 is inserted into outer cannula 2. In an exemplary embodiment of the present invention, the tip 58 of distal end 56 is substantially enclosed and is generally rounded or generally convex. The tip 58 may also be generally blunt. The generally blunt, generally rounded, or generally convex end of the cannula 52 deflects off of dense, cortical bone walls which surround the softer cancellous bone regions, thus re-directing the assembly within the cancellous bone space. This minimizes the risk for accidental perforation of the cortical bone boundaries, which could result in damage to nerves, vessels, or soft tissue structures.

The proximal end of the fenestrated cannula 52 may be secured in a third handle 54. The third handle 54 may be composed of a rigid medical grade plastic or any other rigid medical grade material, such as, for example, stainless steel. A standardized Luer 40 is inset within the third handle 54 to allow attachment of a surgical syringe for bone marrow aspiration.

In one method of using the system of the present invention, the cannulas of the assembled system 1 are aligned such that the distal ends of the cannulas generally overlap in an “open” position (FIGS. 4A and 5C). The mechanical means for locking may lock the handles, consequently aligning the distal ends of the cannulas in the open position. In some embodiments, pin 12 may be inserted into aperture 14 to lock the cannulas in place.

In one method of accessing the cancellous bone space, a cortical drilling device is used to create a hole within the cortex of a bone. Cortical drilling devices are known in the art, per se, and are not a part of the present invention. Once the hole is created, the cortical drilling device is removed and an assembled system 1, wherein the system is in an “open” position, is inserted into the cortical hole.

In preferred methods of inserting the device assembly 1 into the cancellous bone space, a bone marrow aspiration needle assembly comprising an outer cannula and a needle stylet, such as that described in pending U.S. patent application Ser. No. 11/223,085, assigned to the assignee of the present invention, filed on Sep. 9, 2005 and incorporated herein by reference in its entirety, may be used to access the cancellous bone space. Other devices known in the art may also be used. The needle assembly can be used to pierce the cortical wall at the site where bone and bone marrow is to be harvested. After piercing the cortical wall, the needle stylet is withdrawn from the outer cannula. Preferably, a guide wire, for example a Kirschner wire, is inserted through the outer cannula into the cancellous space. The outer cannula is withdrawn and a second cannula is advanced over the guide wire until contact with the distal cortical shell is made. Preferably, the proximal end of the second cannula has an outer diameter greater than the outer diameter of the outer cannula. More preferably, the proximal end of the second cannula has an outer diameter substantially greater than the outer diameter of the outer cannula. In preferred embodiments, the distal end of the second cannula is tapered, such that the outer diameter is substantially equal to the outer diameter of the outer cannula, allowing the distal end of the second cannula to be inserted into the opening in the cortical bone created by the needle assembly. A trephine, or similar instrument, is then advanced over the second cannula to the site where the bone and bone marrow is to be harvested. The trephine is used to create a hole in the cortex of the bone using techniques known in the art. Preferably, with the guide wire in place, the trephine and second cannula are withdrawn and the assembled device of the present invention (1) is advanced over the guide wire and inserted into the pre-formed cortical hole.

Preferably, the entire length of the distal ends of the cannulas is submerged within the cancellous bone. The graduated markings 32 may assist in determining the depth of the system into the cortical hole. The generally blunt, generally rounded, or generally convex ends of the cannulas deflect off of dense, cortical bone walls which surround the softer cancellous bone regions, thus re-directing the assembly within the cancellous bone space. This minimizes the risk for accidental perforation of the cortical bone boundaries, which could result in damage to nerves, vessels, or soft tissue structures.

The mechanical means for locking may be released to unlock the handles, consequently unlocking the distal ends of the cannulas. In some embodiments, pin 12 may be removed from aperture 14 to unlock the handles. The second handle 24 can be rotated around the first handle 4 to cause the inner cannula 22 to rotate within the outer cannula 2 so as to encapsulate a volume of cancellous bone 60 within the distal ends of the cannulas. The cutting surfaces on the distal end of the inner cannula and the rotation of the inner cannula within the outer cannula severs the bone. Angled chamfers 9 and 29 direct the volume of cancellous bone into the interior of the inner cannula. Furrows 38 aid in retaining harvested bone 60 within the cannulas. Preferably, the second handle 24 is rotated approximately 180° relative to the first handle 4 to encapsulate and retrieve a bone sample. In some embodiments, mechanical means such as, for example, 15 and 17, may be used to restrict rotation of the inner cannula when assembled in the outer cannula. In this configuration, the assembly is in a substantially closed configuration to encapsulate a volume of cancellous bone.

The inner cannula 22 may be slidably removed from the outer cannula 2 using the second handle 24. The furrows 38 prevent the volume of cancellous bone 60 from being prematurely dislodged from the inner cannula 22. Once the inner cannula has been removed from the outer cannula, the volume of cancellous bone may be expelled from the inner cannula by tapping the distal end of the inner cannula over a basin 70. Alternatively, the volume of cancellous bone may be expelled by insertion of a plunging device, for example, plunger 50, through a port of the second handle, and into the inner cannula. (See FIG. 7). The plunger should be sufficiently longer that the inner cannula such that the plunger may expel the volume of cancellous bone out of the inner cannula.

In certain embodiments, once a sample of cancellous bone has been harvested, the inner cannula 22 may be re-inserted into outer cannula 2 and the handles locked such that the distal ends of the cannulas are in the open position. The cannulas can be moved as a unit further into the bone to harvest additional cancellous bone samples, using the methods as previously described. These procedures can be repeated as necessary, until sufficient quantities of material have been removed.

Turning to FIGS. 9-11, a fenestrated cannula 52, for example, such as that described herein, can be inserted into the outer cannula 2. Aperture 7 and the semicylindrical shape of the distal end 6 of the outer cannula are such that the fenestrations of the distal end of the fenestrated cannula are exposed within aperture 7. In certain embodiments, the distal tip 58 of the fenestrated cannula 52 may be substantially enclosed. A surgical syringe may be attached to the third handle of the fenestrated cannula via a standardized Luer 40. Bone marrow may then be aspirated from the harvest site using techniques known in the art.

Thus, there have been described presently preferred embodiments of devices and kits for harvesting bone and bone marrow. While the present invention has been particularly shown and described with reference to the presently preferred embodiments thereof, it is understood that the invention is not limited to the embodiments specifically disclosed herein. Numerous changes and modifications may be made to the preferred embodiments of the invention, and such changes and modifications may be made without departing from the spirit of the invention. It is therefore intended that the appended claims cover all such equivalent variations as they fall within the true spirit and scope of the invention.

What is claimed:

1. An apparatus for harvesting bone comprising:
a generally cylindrical outer cannula having one end secured in a first handle and having a distal end that is generally semicylindrical;
a generally cylindrical inner cannula having one end secured in a second handle, the inner cannula having a distal end that is generally semicylindrical;
the inner cannula being rotatable with respect to the outer cannula such that, in cooperation with the outer cannula, a volume at the distal end of both cannulas is substantially enclosed.

2. The apparatus of claim 1 wherein the distal end of the inner cannula further comprises at least one cutting surface.

3. The apparatus of claim 1 wherein the distal ends of the inner and/or outer cannulas have circumferences defined by an arc greater than 180°.

4. The apparatus of claim 1 wherein the distal ends of the inner and/or outer cannulas are defined by an arc greater than about π radians.

5. The apparatus of claim 1 wherein the circumference of the distal ends of the inner and/or outer cannulas are greater than about 50% of the total circumference of the cannulas.

6. The apparatus of claim 1 wherein the circumference of the distal end of the inner cannula is greater than the circumference of the distal end of the outer cannula.

7. The apparatus of claim 1 wherein the inner surface of the distal end of at least one of the cannulas further comprises a plurality of furrows.

8. The apparatus of claim 7 wherein the furrows restrain bone material harvested by the apparatus from prematurely exiting the apparatus.

9. The apparatus of claim 7 wherein the furrows are canted with respect to the transverse plane of the cannulas.

10. The apparatus of claim 7 wherein the furrows form a threaded pattern in the inner surface of the cannulas.

11. The apparatus of claim 1 wherein the first handle and second handle each further comprise at least one port.

12. The apparatus of claim 11 wherein the at least one port of the first handle is configured to receive an inner cannula.

13. The apparatus of claim 11 wherein the at least one port of the first handle is configured to receive a fenestrated cannula.

14. The apparatus of claim 1.1 wherein the at least one port of the second handle is configured to receive a plunging device.

15. The apparatus of claim 1 wherein the first handle and second handle cooperate such that the second handle may be rotated with respect to the first handle causing the distal ends of the cannulas to substantially enclose said volume.

16. The apparatus of claim 1 wherein the outer cannula and inner cannula are coaxial.

17. The apparatus of claim 1 wherein the distal ends of the cannulas further comprise tips at the distal ends that are generally blunt.

18. The apparatus of claim 1 wherein the distal ends of the cannulas further comprise tips at the distal ends that are generally rounded.

19. The apparatus of claim 1 wherein the distal ends of the cannulas further comprise tips at the distal ends that are generally convex.

20. The apparatus of claim 1 wherein the distal ends of cannulas comprise cutting surfaces.

21. The apparatus of claim 1 wherein the distal ends of the cannulas comprise angled chamfers.

22. The apparatus of claim 1 wherein the distal end of the outer cannula further comprises at least one aperture.

23. The apparatus of claim 22 wherein the aperture is elongated.

24. The apparatus of claim 1 wherein the cannulas comprise biocompatible, sterilizable material.

25. The apparatus of claim 1 wherein the handles further comprise cooperating detents.

26. The apparatus of claim 1 further comprising a means for locking the handles.

27. The apparatus of claim 1 further comprising a means for restricting rotation of the inner cannula.

28. The apparatus of claim 1 wherein the cannulas display graduated markings for at least a portion of their length.

29. The apparatus of claim 1 further comprising a cannula having a distal end comprising a plurality of fenestrations.

30. The apparatus of claim 1 wherein the first handle and second handle further comprise a mechanical means for interlocking.

31. The apparatus of claim 1 wherein the first handle further comprises a pin and the second handle further comprises an aperture for receiving said pin.

32. The apparatus of claim 31 wherein the pin and aperture are threaded.

33. A kit for harvesting bone comprising:
a generally cylindrical outer cannula having one end secured in a first handle and having a distal end that is generally semicylindrical; a generally cylindrical inner cannula having one end secured in a second handle, the inner cannula having a distal end that is generally semicylindrical; the inner cannula being rotatable with respect to the outer cannula such that, in cooperation with the outer cannula, a volume at the distal end of both cannulas is enclosed.

34. The kit of claim 33 further comprising a plunger, wherein the plunger may be slidably inserted into the inner cannula.

35. A kit according to claim 33, further comprising a cannula having a distal end comprising a plurality of fenestrations.

36. A kit according to claim 35 wherein the cannula having a distal end comprising a plurality of fenestrations has one end secured in a third handle, wherein the third handle further comprises a Luer fitting.

37. A method for harvesting bone comprising:
piercing a cortical bone using a needle assembly comprising an outer cannula and a needle stylet;
removing the needle sylet from the outer cannula;
inserting a guide wire to the harvesting site;
withdrawing the outer cannula and inserting a second cannula comprising a distal end over the guide wire, such that the distal end enters the cortical bone;
inserting a trephine and operating the trephine to form an opening in the cortical bone;
removing the trephine and second cannula;
inserting an apparatus for harvesting bone comprising a generally cylindrical outer cannula having one end secured in a first handle and having a distal end that is generally semicylindrical; a generally cylindrical inner cannula having one end secured in a second handle, the inner cannula having a distal end that is generally semicylindrical; the inner cannula being rotatable with respect to the outer cannula such that, in cooperation with the outer cannula, a volume at the distal end of both cannulas is substantially enclosed;
rotating the second handle such that the distal ends of the cannulas encapsulate a volume of cancellous bone between the inner cannula and outer cannula; and
removing the volume of cancellous bone by slidably removing the inner cannula from the outer cannula.

38. The method of claim 37 wherein the distal end of the second cannula is tapered.

39. The method of claim 37 further comprising expulsion of the volume of cancellous bone from the inner cannula by insertion of a plunger into the inner cannula.

40. The method of claim 37 wherein the rotation is about 180°.

41. The method of claim 37, further comprising the steps of inserting a cannula having a distal end comprising a plurality of fenestrations into the outer cannula; and aspirating bone marrow.

42. A method for harvesting bone comprising:
inserting a system through a pre-formed cortical opening into cancellous bone;
wherein the system comprises:
a generally cylindrical outer cannula having one end secured in a first handle and having a distal end that is generally semicylindrical;
a generally cylindrical inner cannula having one end secured in a second handle, the inner cannula having a distal end that is generally semicylindrical; the inner cannula being rotatable with respect to the outer cannula such that, in cooperation with the outer cannula, a volume at the distal end of both cannulas is enclosed; and
rotating the second handle such that the distal ends of the cannulas encapsulate a volume of cancellous bone between the inner cannula and outer cannula; and
removing the volume of cancellous bone by slidably removing the inner cannula from the outer cannula.

43. The method of claim 42 further comprising expulsion of the volume of cancellous bone from the inner cannula by insertion of a plunger into the inner cannula.

44. The method of claim 42 wherein the rotation is about 180°.

45. The method of claim 42, further comprising the steps of inserting a cannula having a distal end comprising a plurality of fenestrations into the outer cannula; and aspirating bone marrow.

46. An orthopaedic apparatus comprising:
at least two coaxial cannulas having distal ends;
the distal ends being rotatably cooperative to enclose a volume at said distal ends.
